(19) United States
(12) Patent Application Publication
(10) Pub. No.: US 2009/0117059 A1
(43) Pub. Date: May 7, 2009

(54) COMPOSITIONS AND METHODS OF USE THEREOF, FOR THE TREATMENT OF ORAL PAIN, COMPRISING CLOVES OR EXTRACTS THEREOF IN COMBINATION WITH A STEROID

(76) Inventors: Bryan T. ORONSKY, Los Altos Hills, CA (US); Neil C. ORONSKY, Los Altos Hills, CA (US)

Correspondence Address: MORRISON & FOERSTER LLP, 755 PAGE MILL RD, PALO ALTO, CA, 94304-1018, US

(21) Appl. No.: 12/184,083

(22) Filed: Jul. 31, 2008

Related U.S. Application Data
(60) Provisional application No. 61/007,512, filed on Aug. 3, 2007.
(60) Provisional application No. 61/075,724, filed on Jun. 25, 2008.

(51) Int. Cl. A61K 8/97; A61K 36/61; A61K 31/565; A61Q 11/00; A61P 29/00; A61K 8/34; A61K 31/05

(52) U.S. Cl. 424/58; 424/778; 514/171; 514/734; 424/49

(57) ABSTRACT

Provided are compositions for delivery to an individual's mouth to provide immediate as well as long-lasting pain relief, particularly after tooth extraction or other dental procedure, which include clove, a clove extract or eugenol, and optionally a steroid and other additives including an antibiotic. Kits and systems for delivery of the composition to provide sustained pain relief are also provided.

### CROSS-REFERENCE TO RELATED APPLICATIONS

This application claims priority to U.S. Provisional Patent Application No. 61/007,512, filed on Aug. 3, 2007 and U.S. Provisional Patent Application No. 61/075,724, filed on Jun. 25, 2008, the contents of each of which are incorporated by reference herein in their entirety.

### FIELD OF THE INVENTION

This invention relates to compositions and methods for pain relief, including pain relief related to oral care, wherein the compositions in certain embodiments include cloves or oil of cloves and a steroid. The compositions may for example be in the form of gum, toothpaste, mouthwash, lozenge, floss or mouth spray.

### BACKGROUND

Compositions have been developed in the art for oral care and for oral pain relief. Oral toothpaste compositions comprising calcium carbonate are described in Delany et. al., U.S. Pat. No. 3,935,305.

Compositions comprising clove oil for use as herbal remedies also have been described in the art. Butler, U.S. Pat. No. 5,629,281 relates to a mixture of herbal oils for external application for the treatment of pain associated with minor headaches. The ingredients include the oils of lemon, sweet almond, cajuput, clove, eucalyptus, peppermint and thyme in specific proportions.

Fung et al., U.S. Pat. No. 4,548,809 relates to a method of making a stomatic gargle, by combining eugenol, menthol and eucalyptus oil in ethyl alcohol; licorice extract in water; and sodium monofluorophosphate and sodium fluorite.

Dickey, U.S. Pat. No. 6,353,031 describes a pain relief preparation for use while bathing that includes salts, sodium bicarbonate, clove and nutmeg.

Singh et al., U.S. Pat. No. 6,531,115 describes an analgesic herbal composition including certain essential oils. Kennedy, U.S. Pat. No. 7,166,281, relates to compositions for relieving itch, pain, and swelling associated with insect bites and stings including an abrasive ingredient, a carrier, an enzyme and optionally certain active ingredients, including oils of plants.

Oral compositions including chewing gum compositions comprising diglycerol and other ingredients, additives and filers are described in Stier, U.S. Pat. No. 6,723,304 and Stier, U.S. Pat. No. 6,770,264.

Bhasin, U.S. Patent Application No. 2007/0231274, published Oct. 4, 2007, describes an oral rinse for the treatment of mucositis or stomatitis comprising a corticosteroid. Peyman, U.S. Patent. No. 6,436,429 describes a composition comprising a solid carrier, a bioactive agent, an anti-irritant that is a natural or non-nutrative sweetener.

The area of the mouth presents peculiar challenges to the application of topical pain relievers and ointments because of the naturally moist environment. There is a need for oral compositions useful and suitable for alleviating oral pain.

### SUMMARY

Provided are compositions for the treatment of oral pain as well as methods of use and administration thereof. In certain embodiments, compositions are provided that can be applied to the mouth of an individual to relieve oral pain. In certain embodiments, the compositions provide immediate pain relief.

The composition in one embodiment includes a combination of cloves, oil of cloves, or optionally isolated eugenol, a main ingredient of cloves, and a steroid. While not being limited to any theory, in certain embodiments, the clove component can have the therapeutic effect of providing relatively immediate pain relief, and the steroid can provide longer-lasting pain relief and extend the pain relief effects.

While not being limited to any theory, it is possible that while cloves will give rather immediate relief, the effect is short-lived, approximately one hour, and because of the environment will lose its effectiveness without some added ingredients to extend that pain relief. The steroid is added to extend the life of the pain relief. The various means of administration will allow the effect of the pain relief to be extended and will allow easy application for the user, in certain embodiments, because of the constant application of the compound to the affected area.

Thus, in certain embodiments, cloves or oil of cloves, or eugenol, is used in combination with a steroid, such as a corticosteroid, to provide short term as well as long term pain relief, for example, when administered in an oral topical formulation. A variety of steroids may be used.

Because cloves are bitter, a method to coat or package the clove component with a sweetener should be considered. In order for the individual to tolerate the taste of eugenol, a sweetener may be added to the compound or used in the method of delivery. A variety of different sweeteners may be used, including diglycerol, which is commonly used as a gum sweetener.

An antibiotic may also be included to prevent or lessen the chance of infection in the mouth. Other types of pain relief medications such as lidocaine, benzocaine or novocaine may also be included to give immediate relief.

The composition may be provided in different forms and can be administered in a variety of different manners, such as by directly putting the compound on an individual's finger and having the individual rub the affected gum or tooth. It may also be administered in a gum form, toothpaste, floss, mouth spray, lozenge or mouthwash.

The composition may be administered to patients with oral pain, such as tooth pain, and pain from gums following dental procedures, as wells as patients with bleeding gums or areas in the mouth that are suspect to infection. The composition can be associated with a device or polymer for delivery of the components therein, including gauze, tape, gels and other delivery structures that can be applied to the surface of the mouth. An antibiotic may be added to the device to assist with the prevention of infection.

### DETAILED DESCRIPTION

Compositions including clove, oil of clove, or eugenol and a steroid are provided herein, as well as methods for their use. In certain embodiments, the compositions can provide significant pain relief in a person's mouth both in terms of an immediate effect and a longer lasting effect. In certain embodiments, the principal pain relieving ingredient of the compound is cloves or oil of cloves and is natural.

In certain embodiments, oil of cloves with its main ingredient eugenol is placed in a compound and mixed with a steroid. Oil of cloves from a naturally occurring plant or herb advantageously can be used. It is possible that the oil of cloves will only afford short term pain relief, typically no more than one hour, and therefore, by itself, would need to be constantly applied to the affected area. In order to increase the palliative effect of the pain relief a steroid in a predetermined amount is added to the mixture to extend the pain relieving qualities of the mixture by helping to reduce the pain level and at the same time to reduce the swelling of an affected area. Thus, in order to extend the life and duration of the pain relief, a steroid can be added, which will also have the added benefit of reducing swelling in the mouth, for example, after tooth extraction or gum surgery.

The clove component, such as, clove, oil of clove, or eugenol, in some embodiments comprises about 1 to 50% of the composition, or about 25% to 50%; about 10 to about 30% or about 5 to about 20%. The steroid is optionally about 0.01-25% or about 0.1 to 5%. The clove component and the steroid (and other components as needed) can be compounded for example in standard formulations and carriers known in the art. Examples of carries suited for oral mucosal delivery, include a biocompatible, polyoxyalkylene block copolymer. Exemplary polyoxyalkylene block copolymers for use in delivery compositions for delivery are polyoxyethylene-polyoxypropylene block copolymers. Pluronic gels can be used. Examples of some polyoxyalkylene block copolymers include Pluronic™ F68 (a poloxamer 188), Pluronic™ F127 (a poloxamer 407), Pluronic™ L121 (a poloxamer 401), and Pluronic™ L101 (a poloxamer 331), and Tetronic™ T1501 (a poloxamine). Pluronic™ and Tetronicυ are trademarks of BASF Corporation. Furthermore, more than one of these and/or other polyoxyalkylene block copolymers may be included in the delivery composition. Also, other polymers and/or other additives may also be included in the delivery composition to the extent the inclusion is not inconsistent with the desired characteristics of the delivery composition. Furthermore, these polymers may be mixed with other polymers or other additives, such as sugars, to vary the transition temperature range, typically in aqueous solutions, at which reverse-thermal viscosity behavior occurs.

Bioadhesive carriers known in the art that can be used include gels, pastes, tablets, and films. For example, U.S. Pat. Nos. 5,192,802, 5,314,915, 5,298,258, and 5,642,749 describe bioadhesive gels. Denture adhesive pastes are described in, for example, U.S. Pat. Nos. 4,894,232 and 4,518,721. A commercial product under the name Orabase™-B, which is a thick gel or paste for the relief of mouth sores, is another example of an adhesive paste that can be used as a carrier. A dental paste may contain components such as gelatin, pectin, and carboboxymethylcellulose sodium in Plastibase® (Plasticized Hydrocarbon Gel), a polyethylene and mineral oil gel base. Bioadhesive tablets are described in U.S. Pat. Nos. 4,915,948, 4,226,848, 4,292,299, and 4,250,163, as having single layer or bilayers. U.S. Pat. Nos. 3,996,934 and 4,286,592 describe the use of bandages or bioadhesive laminated films. U.S. Pat. Nos. 4,517,173, 4,572,832, 4,713,243, 4,900,554, and 5,137,729 describe delivery systems for use on mucosal surfaces. Compositions made of hydroxypropyl cellulose can be used. The patents and patent applications referred to herein are incorporated herein by reference.

A plurality of steroids may be used to extend the pain relief. For instance more than one steroid may be added to the cloves so that when the pain relief of the cloves begins to wear off a steroid will activate to extend the pain relief a predetermined amount of time. Additionally other steroids may also be included to further extend the pain relief. Although cloves is a principal pain relief ingredient, the use of more than one steroid to extend the pain relief is not precluded in this application.

In one embodiment, the steroid is a glucocorticoid steroid. In another embodiment, the steroid has anti-inflammatory activity. Exemplary steroids include 21-acetoxypregnenolone, alclometasone, algestone, amcinonide, beclomethasone, betamethasone, budesonide, chloroprednisone, clobetasol, clobetasone, cloprednol, clocortolone, corticosterone, cortisone, cortivazol, deffazacort, desonide, desoximetasone, dexamethasone, diflucortolone, diruprednate, enoxolone, fluazacort, flucloronide, flumethasone, flunisolide, fluocinolone acetonide, fluocinonide, fluocortinbutyl, fluocortolone, fluorometholone, fluperolone acetate, fluprednidene acetate, fluprednisolone, flurandrenolide, formocortal, halcinonide, halometasone, halopredone acetate, hydrocortamate, diflorasone, hydrocortisone, hydrocortisone acetate, hydrocortisone phosphate, hydrocortisone 21-sodium succinate, hydrocortisone tebutate, mazipredone, medrysone, meprednisone, methylprednisolone, mometasone furoate, prednisolone sodium 21-m-sulfobenzoate, prednisolone 21-stearoylglycolate, prednisolone tebutate, prednisolone 21-trimethylacetate, prednisone, prednival, paramethasone, prednylidene, prednicarbate, prednylidene 21-diethylaminoacetate, prednisolone, prednisolone 21-diethylaminoacetate, tixocortol, triamcinolone, prednisolone sodium phosphate, progesterone, triamcinolone acetonide, prednisolone sodium succinate, triamcinolone benetonide, triamcinolone hexacetonide, and mixtures thereof. Optionally a plurality of steroids are used. In certain embodiments, the steroid is triamcinolone (KENALOG).

In certain embodiments, the steroid that is present in an amount that is a maximum of 25 mg, about 20 mg, about 15 mg, about 10 mg, about 5 mg, about 3 mg, about 1 mg or 0.5 mg, per 100 g.

Since the main ingredient of cloves or eugenol is bitter tasting, a sweetener can be added. A common sweetener for gum is diglycerol and that would be a suitable choice of sweetener. Additionally cloves may also be wrapped in a piece of candy that will offset the taste of the cloves.

In another embodiment, lidocaine, novocaine, benzocaine or a narcotic may be added for more immediate pain relief in the mouth.

The compound can be placed or applied directly on the affected gum or tooth by placing it on someone's finger and rubbing the tooth or affected gum area. The compound may be administered in a paste, gum, toothpaste, mouthwash, mouth spray, paste, lozenge or coated floss form. The benefit of applying this compound in the form of gum, in particular, is that the affected area would be constantly exposed to the compound. Other means to administer may also be used.

Additionally in the case of an oral application, nicotine may be added to a gum to provide the added benefit of decreasing smoking in certain patients. Nicotine is a well known vasoconstrictor and hastens tooth decay and overall poor oral health in general.

Because of the environment in the mouth and its challenges in applying any type of external pain relief compound and additionally because of the added risk of infection in the mouth following a tooth extraction, for instance, an antibiotic may also be added to the compound to assist with the control of infection. Many different antibiotics may be used.

Exemplary antibiotics include include β-lactam antibiotics, macrolides, monobactams, rifamycins, tetracyclines, chloramphenicol, clindamycin, lincomycin, fusidic acid, novobiocin, fosfomycin, fusidate sodium, capreomycin, colistimethate, gramicidin, minocycline, doxycycline, bacitracin, erythromycin, nalidixic acid, vancomycin, and trimethoprim. Exemplary β-lactam antibiotics include ampicillin, aziocillin, aztreonam, carbenicillin, cefoperazone, ceftriaxone, cephaloridine, cephalothin, cloxacillin, moxalactam, penicillin G, piperacillin, and ticarcillin. Other antibiotics including cation peptides also are suitable.

The composition may further include an additive, such as an oral disinfectant, cleansing or antiplaque agent. Exemplary additives include sodium bicarbonate, phosphoric acid, hydrogen peroxide, triclosan, chlorhexidine gluconate, sodium lauryl sulfate, acetic acid, adipic acid, ascorbic acid, citric acid, dehydroacetic acid, erythorbic acid, fumaric acid, glutaric acid, gluconic acid, hyaluronic acid, hydroxyacetic acid, lactic acid, malic acid, polymerized carboxylic acids comprising polylactic or polylactic-glycolic acids, succinic acid, sulfamic acid, tannic acid, tartaric acid, or mouthwashes such as Listerine.

In certain embodiments, the composition comprises a flavorant such as peppermint, lemon, spearmint, wintergreen, cinnamon, apple, cherry, lime, orange, peach, apricot, pear, melon, kiwi, passionfruit, or any natural or synthetic flavorant. Other flavorants include sweeteners such as lactose, glucose, maltose, sorbitol, and sodium cyclamate.

In one embodiment, a composition is provided that comprises oil of cloves, lidocaine, triamcinolone, bicarbonate and peppermint.

The compositions may be administered to an individual's mouth to provide immediate as well as long-lasting pain relief, particularly after tooth extraction or other dental procedure. In certain embodiments, the methods and compositions permit the administration of the composition to allow it to persist and remain over time in the oral mucosa to allow the therapeutic pain relief. In one embodiment, the composition persists in the mouth to allow pain relief, for example, for at least 5, 10, 15, 20, 30, 40 minutes or at least one, two, three, four, five, six, seven, eight, nine, ten, eleven, twelve or thirteen hours.

In certain embodiments, after treatment of patients after tooth extraction, the affected area of pain can decrease substantially for example within one hour with the application of the compound without steroid, and, e.g., for about twelve to fourteen hours, with steroid added.

In certain embodiment, formulations with the following percentages are provided.

By way of example, 60 g, of mouth cream contains:
Kenalog 0.1% (60 mg)
clove oil 0.5% (0.3 g)
sodium bicarbonate 4.5% (2.7 g)
Orabase qs (which contains 20% benzocaine) until 60 g is reached.

Orabase is commercially available and contains 20% benzocaine as well as inactive ingredients including cellulose gum, flavor, pectin, plasticized hydrocarbon gel, preservative, and xanthine gum.

In another embodiment 30 g of mouth cream contains:
Kenalog 0.1% (0.03 g)
clove oil 0.5% (0.15 ml or 0.15 g)
sodium bicarbonate 4.5% (1.35 g)
Stevia 0.1% (sweetener, 30 mg)
acesulfam potassium 0.1% (30 mg)
Lidocaine USP 5%=1.5 g
Lidocaine HCl (1.5 g)
Lecithin isopropyl palmitate solution (6.6 ml)
pluronic 30% gel qs (about 21.5 g, to bring to 30 g)

The composition can be used, for example, on patients with odontogenic infections, gingivitis, denture pain from poor fitting and from acute pressure due to chewing, teeth extractions, canker sores, cold sores, teething pain and sore throats.

An antibiotic may be included in the mixture to assist in fighting infection in the mouth. The compositions also can be used to treat mouth sores or other places in the mouth that require pain relief.

In certain embodiments, sweeteners such as Stevia, and sodium saccharide can be added to the composition.

The composition can be for example in a form such as a gel, which optionally promotes penetration of actives. Thus, the compositions in certain embodiments are administered in a transdermal or transmucosal fashion through a gel such as a PLURONIC gel. PLURONIC gels include commercially available polyoxyethylene-polyoxypropylene block copolymers.

In certain embodiments, the composition can include a mucoadhesive polymer. Mucoadhesive polymers that can be used include hydrophilic polymers and hydrogels. Hydrophilic polymers include those containing carboxylic groups. Examples of polymers include poly vinyl pyrrolidone (PVP), methyl cellulose (MC), sodium carboxy methylcellulose (SCMC), hydroxy propyl cellulose (HPC) and other cellulose derivatives. Hydrogels typically will swell by absorbing water interacting by means of adhesion with the mucus in the mouth. Examples include those with anionic, cationic and neutral groups, such ascarbopol, polyacrylates and crosslinked forms, chitosan and its derivatives and EUDRAGIT polymers.

Further, a lectin polymer may be used, or a copolymer of PAA and PEG monoethylether monomethacrylate(PAA-co-PEG) (PEGMM), such as a Corplex™ PSA adhesive hydrogel. Such hydrogels are prepared for example by non-covalent (hydrogen bond) cross-linking of a film-forming hydrophilic polymer(for example PVP) with a short-chain plasticiser (typically PEG) with complementary reactive hydroxyl groups at the chain ends. An AB block copolymer of oligo(methyl methacrylate) and PAA (polyacrylamide) also can be used.

In one embodiment, a pluronic gel is provided in the composition, e.g., in amounts of about 30-70%, e.g., 60% to get the consistency of a paste.

The compositions may be provided in a mucoadhesive matrix-forming polymer or polymer combination. Water-soluble or partially water-soluble polymers, not excluding any other suitable raw materials, include polyvinyl alcohol; cellulose derivatives such as hydroxypropyl methyl cellulose, hydroxypropyl cellulose, sodium carboxymethyl cellulose, methyl cellulose, hydroxyethyl cellulose and hydroxypropyl ethyl cellulose; starch and starch derivatives; gelatine; polyvinyl pyrrolidones; gum arabic; pullulan; and acrylates. In addition, polymers from the following group are suitable as water-soluble or swellable polymers: dextran; cellulose derivatives, such as carboxymethyl cellulose and ethyl or propyl cellulose; polyacrylic acid, polyacrylates, polyethylene oxide polymers, polyacrylamides, polyethylene glycol, collagen, alginates, pectins, tragacanth, chitosan, alginic acid, arabinogalactan, galactomannan, agar-agar, agarose, carrageenan, and natural gums.

The composition can be administered for example by a medical professional or by the individual in need thereof. In certain embodiments, the compositions may be provided in a carrier or device that facilitates administration. Exemplary devices include mouth strips, polymers, dentures, a deformable mold, chewing gum, and other devices that remain in the mouth and allow for release of active over a period of time, e.g. 1, 2, 3, 4, 5, 6, 7, 8, 9, 10, 11, 12, 13, 14 or more hours. Other devices include teeth caps and bridges that can be loaded with an effective amount of the compositions disclosed herein. The compositions also can be provided on devices such as coated floss, dental picks, toothpicks, swabs, toothbrushs, a travel toothbrush through which stored cream can be dispensed, burstable blister packs which can be placed in the mouth releasing preformed amounts on demand, pre-loaded applicators with preformed amounts, coated dentures, dissolvable tablets, gargle tablets, foodstuffs, or bandages that can be placed on the skin of the face over an area of swelling or edema or over the temporo-mandibular joint for TMJ, and lip balm wherein the cream is spread over the lips and the tongue then serves as the applicator.

The compositions and devices also can be used for relief of pain from dental restorations involving formulations of amalgams, cementations, impressions, composites, composite restorations/sealants, or crown and bridge preparations which have been newly placed in different areas in the patient's mouth and pain from teeth whitening and pain from gingival recession, clenching or bruxing, pain from cellulitis like Ludwig's angina, pain from facial edema, pain from temporo-mandibular joint disease (TMJ), pain from cold sores, and pain from thrush. The compositions and devices also can be used for the treatment of cold sores (herpes labialis) and thrush, and the compositions also can include acyclovir or pencyclovir, for example, in the case of herpes. The compositions also can include an antifungal like nystatin for example for the treatment of thrush. The compositions also can contain various antioxidants and vitamins, such as folic acid, and vitamin C where vitamin C deficiency has occurred, e.g. in association with scurvy. The compositions and devices may be used to treat patients with extremely poor dental hygiene who may be vitamin deficient, such as alcoholics or drug abusers. The compositions in one embodiment may contain antabuse or metronidazole (Flagyl) antibiotic which is associated with antabuse-like effects when drinking alcohol.

In certain embodiments, the compositions may include a sweetener such as sugar. The compositions may be used to treat sore gums, and may include vitamins, such as vitamin E, antioxidants or herbals that are optionally soothing.

In certain embodiments, the composition is delivered directly to the mouth, e.g. by a spray or mechanically, e.g., with a cloth, a swab (such as a cotton swab), a sponge, a gummy material, or with a finger. Gels such as pluronic gels, molds, gums and other mouth devices also can be loaded or reloaded with the effective compositions, for example, through the use of a spray or syringe.

The compositions also may further include an antiviral, antibiotic or antibody against bacteria in an effective amount to reduce the chance of infection, and can be formulated for use on dental equipment or application to the mouth during a dental procedure, to, for example, help reduce the risk of endocarditis. Thus in some embodiments methods, compositions and devices for reducing the risk of heart infection during dental procedures are provided.

Examples of suitable vehicles or devices for use in administering the compositions include those described in: 1) U.S. Pat. No. 2,430,740 which relates to mixing a fibrous mineral medicament with therapeutically inert fibers, such as cotton fibers, to form a therapeutic dressing; 2) U.S. Pat. No. 5,192,802 which relates to a bioadhesive pharmaceutical carrier containing a polymer blend of sodium carboxymethyol cellulose and xanthan gum or sodium alginate for use as an oral bioadhesive pharmaceutical carrier; 3) U.S. Pat. No. 4,876,092 relating to an adhesive preparation including an adhesive layer containing carboxyvinyl polymer, a water-insoluble methacrylic copolymer, a polyhydric alcohol and a pharmaceutically active agent, and a water-impermeable and water-insoluble carrier layer containing a plasticizer which can adhere to a patient's oral cavity; 4) U.S. Pat. No. 4,981,875 relating to the use of etofenamate for preparing compositions to treat bacterial, viral or fungicidal inflammations in a patient's oral cavity; 5) U.S. Pat. No. 5,242,910 relating to the use of polylactide/glycolide compositions for releasing drugs in a patient's oral cavity; 6) U.S. Pat. No. 3,219,527 relating to eugenol-containing periodontal dressings; 7) U.S. Pat. No. 5,437,872 relating to a pharmaceutical tablet or lozenge comprising a non-absorbable pharmaceutically active agent combined with a tablet matrix for controlled and sustained release of the agent into a patient's mouth and gastro-intestinal tract; and 8) U.S. Pat. No. 5,102,666 relating to a polymeric delivery system for controlled intra-oral release of active agents, such as medicinal agents, breath fresheners and flavors, where the active agent is combined with a calcium polycarbophil matrix.

In some embodiments, the surface of the dressing is in contact with the affected area and is available to deliver the medication, for example, for a few hours or longer. A surgical cement pack can be used that is applied directly to an affected intra-oral area while it is soft and moldable, wherein the dressing hardens once it comes into contact with water. A water-soluble alginate salt, such as sodium or potassium alginate, can be reacted in water with a calcium or lead salt to form a water-insoluble gel dental impression material.

Flexible intra-oral bandages capable of delivering medication in a water-washed environment over an extended period of time, which remains securely in place in a patient's mouth, will not irritate surrounding healthy gum and mouth tissue and can be easily changed by the patient also can be used, as well as a kit for such an intra-oral bandage. The bandage can comprise a flexible, cohesive hydrolyzed gel/water-wettable, fiber-reinforced material. A kit for making the bandage can also be used, comprising a hydrolyzable powder/water-wettable fiber mixture enclosed in a flexible, water-permeable, non-stick envelope that does not adhere to the hydrolyzed gel/fiber product after wetting with an aqueous liquid. The hydrolyzable powder is optionally a water-soluble alginate salt, commonly used for forming dental impressions mixed with another salt which forms a water-insoluble alginate gel in water. The envelope is for example a non-woven water-permeable fabric. Optionally, the hydrolyzable powder/water-wettable fiber mixture and water-permeable envelope kit is wrapped in a package having a non-adherent surface, such as a package formed of perforated aluminum foil and Mylar film. The package may also be water-permeable.

To activate the bandage, the powder/water-wettable fiber mixture enclosed in the water-permeable envelope is immersed in an aqueous liquid, such as water. Alternatively, where the outer package is water-permeable, the water-permeable package containing the hydrolyzable powder/water-wettable fiber mixture enclosed in the envelope is soaked in the aqueous liquid. The water-permeable envelope (and the water-permeable package) allows the liquid to pass through to the hydrolyzable powder-water wettable fiber mixture to wet the mixture and convert it to a moldable tacky fiber-reinforced gel. The envelope also serves to retain the alginate powder-water wettable fiber mixture while immersed in any aqueous liquid.

The “hydrolyzable powder” may be particulate, i.e., non-fibrous, in nature, having a particle size relatively substantially smaller than the length of the wettable fibers, and the powder can include short, fibrous, hydrolyzable particles, preferably which are much shorter in length than the wettable fibers.

The wettable fibers can for example have an individual length of at least 3 mm, or in the range of from about 2 mm to about 4 mm, to obtain the desired reinforcement effect. There can in some embodiments be at least one order of magnitude difference between the diameter and length of the wettable fibers. The fibers can be provided loose or by way of a preformed tissue sheet.

After the kit is removed from the liquid, the tacky fiber-reinforced gel is removed from the package and envelope, and manually molded and positioned in place over a desired tissue surface in a patient's oral cavity.

The flexible intra-oral bandage can be loaded with active and the bandage and the kits therefor can be capable of delivering actives in a water-washed environment for an extended period of time while remaining securely in place in a patient's mouth without irritating surrounding tissue.

See, e.g., exemplary delivery vehicles described in U.S. Pat. No. 6,146,655, the disclosure of which is incorporated herein, which describes, in one embodiment, a composition of a hydrolyzable powder including: about 12 weight percent sodium or potassium alginate; about 12 weight percent of a reactor salt, such as calcium sulfate hemihydrate; about 70 weight percent diatomaceous earth; about 2 weight percent tri-sodium phosphate; and about 1 weight percent corn starch.

A variety of kits are possible including, by way of example, a container that includes the active composition and a swab. The composition also may be provided in a spray device. Materials and devices can be preloaded with an effective amount of the composition and optionally be refillable, e.g., by adding more active via a spray or syringe mechanism.

In certain embodiments, sonophoresis or electrophoresis can be used to enhance permeation.

The compositions and/or devices also can be used in conjuction with a patient-controlled device or procedure for inhibiting intraoperative dental pain. For example devices or methods can be used that take advantage of the ‘Gate Theory’ of sensation, principally painful sensation (Melzack R, Wall P D: Pain mechanisms: A new theory. Science 150:971, 1965), where it was proposed that input signals from painful stimuli, on reaching the spinal nerves, via normal connecting nerve fibers, are subjected to an anatomical ‘gate’ or switch in a specific part of the spinal cord. This gate, by incoming signal type and intensity, determined if the signals would be passed up the spinal cord to the sensory areas of the brain or locally blocked. Painful stimuli of sufficient intensity and arising from specific areas from peripheral injuries are passed by this ‘gate’ if unopposed by other sensory input; this mechanism alerts the patient to potential or actual tissue injury. Non-painful stimuli of sufficient amplitude and from similar locations as the pain source could ‘close’ the ‘gate’ to the painful signals, blocking their transmission upward to conscious experience. A variety of devices can be used to provide non-painful stimuli of sufficient amplitude. The device in one embodiment may comprise a removable tooth clamp; an actuator unit including a vibratory actuator and a coupling device for coupling the vibrating actuator to the tooth clamp; and a control box electrically connected to the vibratory actuator and including at least one controller; wherein the removable tooth clamp is configured to be attached to at least one tooth of a patient, and further wherein upon final assembly the at least one controller initials oscillation of the tooth clamp via the vibratory actuator, vibrating the at least one tooth to inhibit oral dental pain. See, e.g. U.S. 2006/0275739A.

Individuals that can be treated include humans, but also pets and other domestic animals suffering from oral pain, including dogs, cats and horses.

### EXAMPLES

### Example 1

A patient with a periapical abscess complained of severe localized pain and swelling over a lower molar with thermal sensitivity. A paste comprised of 0.1% kenalog, 0.5% cloves and 4.5% sodium bicarbonate with peppermint flavoring in Orabase was applied to a Q-tip and swabbed over the affected tooth. After 1 minute the patient reported total numbness and relief of pain which lasted for 1 hour. The pain returned but with a lesser intensity. 3 hours later the patient swabbed more paste over the tooth with similar results. Orabase is commercially available and contains 20% benzocaine as well as cellulose gum, flavor, pectin, plasticized hydrocarbon gel, preservative, and xanthine gum.

### Example 2

A patient presented with trench mouth characterized by painful edematous, and ulcerated gingiva. A small amount of paste comprised of 0.1% kenalog, 0.5% cloves and 4.5% sodium bicarbonate with peppermint flavoring (in Orabase with 20% benzocaine) was applied to a Q-tip and swabbed over the gums with total relief for about 1 hour. The pain returned gradually thereafter reaching a renewed peak about 8 hours later at which time the patient applied more paste with similar results.

### Example 3

The patient presented with a superficial dental infection complaining of localized pain, edema, and sensitivity to temperature and air. A small amount of paste comprised of 0.1% kenalog, 0.5% cloves and 4.5% sodium bicarbonate with peppermint flavoring (in Orabase with 20% benzocaine) was applied to a Q-tip and swabbed over the gums with total relief for about 30 minutes at which time the pain returned but at a lesser intensity. The pain again reached a crescendo about 2 hours later at which time the paste was applied with similar results.

While certain embodiments of the invention have been disclosed, certain modifications may be made by those skilled in the art to modify the invention without departing from the spirit of the invention.

What is claimed is:

1. A method of alleviating pain in an individual in need thereof, the method comprising administering a pain relieving oral agent composition to the interior of the mouth of the individual, the composition comprising:
a) cloves, a clove extract, or eugenol;
b) a steroid;
c) optionally an antibiotic; and
d) optionally a pain relieving agent.

2. The method of claim 1, wherein the composition comprises eugenol and a steroid.

3. The method of claim 1, wherein the composition comprises cloves.

4. The method of claim 1, wherein the composition comprises oil of cloves and a steroid.

5. The method of claim 1 wherein the steroid is selected from hydrocortisone and cortisone, and triamcinolone.

6. The method of claim 1 wherein the antibiotic is selected from β-lactam antibiotics, macrolides, monobactams, rifamycins, tetracyclines, chloramphenicol, clindamycin, lincomycin, fusidic acid, novobiocin, fosfomycin, fusidate sodium, capreomycin, colistimethate, gramicidin, minocycline, doxycycline, bacitracin, erythromycin, nalidixic acid, vancomycin, and trimethoprim.

7. The method of claim 1 wherein the pain relieving agent is benzocaine, lidocaine, novocaine or a narcotic medication.

8. The method of claim 1 wherein the composition is in the form of a gum, toothpaste, mouthwash, mouth spray, paste, floss, or candy.

9. The method of claim 1 wherein the composition is administered transdermally.

10. The method of claim 1 wherein the composition is administered transmucosally.

11. The method of claim 1 wherein the composition comprises a sweetener.

12. The method of claim 1 wherein the composition comprises nicotine.

13. The method of claim 1 wherein the composition further comprises an additive selected from sodium bicarbonate, phosphoric acid, hydrogen peroxide, triclosan, chlorhexidine gluconate, sodium lauryl sulfate, acetic acid, adipic acid, ascorbic acid, citric acid, dehydroacetic acid, erythorbic acid, fumaric acid, glutaric acid, gluconic acid, hyaluronic acid, hydroxyacetic acid, lactic acid, malic acid, a polylactic or polylactic-glycolic acid, succinic acid, sulfamic acid, tannic acid, and tartaric acid.

14. The method of claim 1 wherein the composition further comprises a flavorant.

15. The method of claim 14 wherein the flavorant is selected from peppermint and spearment.

16. The method of claim 1 wherein the composition persists in the mouth to allow pain relief.

17. The method of claim 1, wherein the pain relieving components of the composition persist in the mouth and alleviate pain for at least 5, 10, 15, 20, 30, or 40 minutes, or at least 1, 2, 3, 4, 5, 6, 7, 8, 9, 10, 11, or 12 hours.

18. A delivery system for alleviating oral pain in the mouth of an individual in need thereof comprising:
a) a composition comprising:
i) cloves, a clove extract, or eugenol;
ii) a steroid;
iii) optionally an antibiotic; and
iv) optionally a pain relieving agent; and
b) a device or material for application of the composition to the individual in an effective amount and for an effective time to alleviate pain.

19. The system of claim 18, wherein the device or material is selected from a spray device, applicator, polymer, gum, dental tray or dental tape.

20. The system of claim 18, wherein the device or material is a mucoadhesive polymer material.

21. The system of claim 20, wherein the polymer material is selected from polyoxyethylene-polyoxypropylene block copolymers, poly vinyl pyrrolidone (PVP), methyl cellulose (MC), sodium carboxy methylcellulose (SCMC), hydroxy propyl cellulose (HPC), ascarbopol, polyacrylates forms, chitosan polymers, and lectins.

22. The system of claim 18, wherein the composition comprises eugenol and a steroid.

23. The system of claim 22, wherein the steroid is selected from hydrocortisone and cortisone, and triamcinolone.

24. The system of claim 22, further comprising means for reloading the device or material with the composition.

25. The system of claim 24, wherein the individual's pain is relieved for at least 5 mins, 10 mins, 15 mins, 20 mins, 30 mins, 40 minutes, one hour, two hours, three hours, four hours, five hours, six hours, seven hours, eight hours, nine hours, ten hours, eleven hours, or twelve hours after application.
